(19) United States
(12) Patent Application Publication
(10) Pub. No.: US 2026/0277240 A1
(43) Pub. Date: Sep. 17, 2026

(54) TRANSPORT ROBOT, SURGICAL ROBOT SYSTEM INCLUDING THE SAME, AND METHOD

(71) Applicant: LIVSMED INC., Seongnam-si (KR)

(72) Inventors: Jae Hoon SHIN, Gwangju-si (KR); Gyu Dong JEON, Seoul (KR); Soon Yong JEONG, Seongnam-si (KR); Chang Wha JUNG, Pohang-si (KR)

(21) Appl. No.: 19/562,180

(22) Filed: Mar. 10, 2026

(30) Foreign Application Priority Data
Mar. 12, 2025 (KR) 10-2025-0032014

(51) Int. Cl. G05D 1/667; A61B 34/30; G05D 107/60

(52) U.S. Cl. CPC G05D 1/667; A61B 34/30; G05D 2107/65

(57) ABSTRACT

A transport robot, a surgical robot system including the transport robot, and a method are provided. More particularly, a surgical robot system including a surgical robot used in laparoscopic surgery or various other surgeries and a transport robot configured to transport the surgical robot is provided, and a method of transporting and placing the surgical robot using the transport robot is provided. The surgical robot system includes a surgical robot including at least one arm unit, a transport robot operable to transport the surgical robot, and a master robot including a manipulation member configured to control a motion of the transport robot, wherein the transport robot moves toward the surgical robot in response to a received signal and places the surgical robot at a position corresponding to the received signal.

### CROSS-REFERENCE TO RELATED APPLICATION

This application is based on and claims priority under 35 U.S.C. § 119 to Korean Patent Application No. 10-2025-0032014, filed on Mar. 12, 2025, in the Korean Ministry of Intellectual Property (MOIP) of the Republic of Korea, the disclosure of which is incorporated by reference herein in its entirety.

### BACKGROUND

### 1. Field

The present disclosure relates to a transport robot, a surgical robot system including the transport robot, and a method, and more particularly, to a surgical robot system including a surgical robot used in laparoscopic surgery or various other surgeries and a transport robot configured to transport the surgical robot, and to a method of transporting and placing the surgical robot using the transport robot.

### 2. Description of the Related Art

In medical terms, surgery refers to treatment of a disease by cutting, incising, or manipulating the skin, mucous membranes, or other tissues using medical devices. In particular, open surgery, in which the skin at a surgical site is incised and opened to treat, reconstruct, or remove internal organs, may cause problems such as bleeding, side effects, patient pain, and scarring. Accordingly, in recent years, as alternatives, there has been increasing interest in surgeries performed by making one or more small openings in the skin and inserting medical devices such as a laparoscope, surgical instruments, and an operating microscope for microsurgery, and in surgeries performed using robots.

Here, a surgical robot refers to a robot having functions capable of replacing surgical procedures traditionally performed by a surgeon. Such surgical robots are capable of performing operations more accurately and precisely than a human and have the advantage of enabling remote surgery.

Surgical robots currently under development worldwide may include a bone surgery robot, a laparoscopic surgery robot, a stereotactic surgery robot, and the like. Here, the laparoscopic surgery robot is a robot that performs minimally invasive surgery using a laparoscope and small surgical instruments.

Meanwhile, a surgical robot generally includes a master robot and a slave robot. When a surgeon manipulates a control lever (for example, a handle) provided on the master robot, a surgical tool coupled to a robot arm of the slave robot or held by the robot arm is manipulated to perform surgery.

Meanwhile, in a slave robot having two or more robot arms, vibration generated in one robot arm during operation of a surgical instrument may be transmitted to another robot arm, and such vibration may affect control performance of the slave robot. Accordingly, in order to minimize vibration of the robot arms, stiffness and mass of a base or a frame are increased.

However, when the stiffness and mass of the frame increase, the weight of the robot also increases. As a result, when a user attempts to move the slave robot to change the placement, moving the slave robot becomes difficult and inconvenient, and a problem may occur in which the slave robot is not accurately placed at a location where the slave robot is required to be placed.

The background art described above is technical information possessed by the inventors for developing the present disclosure or acquired by the inventors in the course of developing the present disclosure, and thus is not necessarily publicly known or publicly available prior art before the filing of the present application.

### SUMMARY

The present disclosure has been made in view of the above-described problems, and an object thereof is to provide a surgical robot system that improves ease of moving a surgical robot while allowing the surgical robot to be lightweight, and that is capable of automatically placing the surgical robot. However, such an object is merely exemplary, and the scope of the present disclosure is not limited thereby.

According to an aspect of the present disclosure, there is provided a surgical robot system including a surgical robot including at least one arm unit, a transport robot operable to transport the surgical robot, and a master robot including a manipulation member configured to control a motion of the transport robot, wherein the transport robot moves toward the surgical robot in response to a received signal and places the surgical robot at a position corresponding to the received signal.

In an embodiment of the present disclosure, the transport robot may include a traveling part configured to move or stop a body of the transport robot, a surgical robot connection part configured to be brought into contact with and connected to the surgical robot, and a detection part configured to detect a surrounding environment of the transport robot and assist a travel of the transport robot.

In an embodiment of the present disclosure, the surgical robot connection part may include a lifting module configured to raise and lower the surgical robot, and the transport robot may be further operable to transport the surgical robot while the surgical robot is lifted by the lifting module.

In an embodiment of the present disclosure, the surgical robot connection part may include a docking module configured to be connected to the surgical robot, and the transport robot may be further operable to move the surgical robot by pulling or pushing the surgical robot while the transport robot is connected to the surgical robot via the docking module.

In an embodiment of the present disclosure, the detection part may be operable to detect the surgical robot in a vicinity of the transport robot.

In an embodiment of the present disclosure, the detection part may be operable to detect a surgical bed positioned in a surgical space.

In an embodiment of the present disclosure, the transport robot may further include a power supply part configured to supply a power to the surgical robot.

In an embodiment of the present disclosure, the surgical robot system may further include a surgical bed including a sensor configured to transmit position information of the surgical bed to at least one of the surgical robot, the transport robot, and the master robot.

In an embodiment of the present disclosure, the master robot may be operable to control a motion of the surgical robot.

In an embodiment of the present disclosure, the transport robot may be further operable to autonomously travel in an operating room.

In an embodiment of the present disclosure, the transport robot may place the surgical robot at a preset position in response to the received signal.

In an embodiment of the present disclosure, position information indicating a position at which the surgical robot is placed may be stored while being categorized according to a type of surgery, and the transport robot may place the surgical robot according to a type of surgery selected among stored types of surgery.

In an embodiment of the present disclosure, the surgical robot system may include an autonomous traveling mode and a travel assist mode among selectable driving modes for the transport robot.

In an embodiment of the present disclosure, when the autonomous traveling mode is selected, the transport robot may perform an autonomous traveling function to move toward the surgical robot, may be connected to the surgical robot, and may place the surgical robot at a preset position.

In an embodiment of the present disclosure, when the travel assist mode is selected, the transport robot may detect a force transmitted thereto from outside to drive the traveling part.

According to another aspect of the present disclosure, there is provided a transport robot for transporting a surgical robot, including a traveling part configured to move or stop a body of the transport robot, a surgical robot connection part configured to be brought into contact with and connected to the surgical robot, and a detection part configured to detect a surrounding environment of the transport robot and assist a travel of the transport robot, wherein the transport robot moves toward the surgical robot in response to a received signal and places the surgical robot at a position corresponding to the received signal.

In an embodiment of the present disclosure, the surgical robot connection part may include a lifting module configured to raise and lower the surgical robot, and the transport robot may be further operable to transport the surgical robot while the surgical robot is lifted by the lifting module.

In an embodiment of the present disclosure, the surgical robot connection part may include a docking module configured to be connected to the surgical robot, and the transport robot may be further operable to move the surgical robot by pulling or pushing the surgical robot while the transport robot is connected to the surgical robot via the docking module.

In an embodiment of the present disclosure, the detection part may be operable to detect the surgical robot in a vicinity of the transport robot.

In an embodiment of the present disclosure, the detection part may be operable to detect a surgical bed positioned in a surgical space.

In an embodiment of the present disclosure, the transport robot may be operable to receive position information of a surgical bed from the surgical bed.

In an embodiment of the present disclosure, the transport robot may further include a power supply part configured to supply a power to the surgical robot.

In an embodiment of the present disclosure, the transport robot may be operable to autonomously travel in an operating room.

In an embodiment of the present disclosure, the transport robot may place the surgical robot at a preset position in response to the received signal.

In an embodiment of the present disclosure, position information indicating a position at which the surgical robot is placed may be stored while being categorized according to a type of surgery, and the transport robot may place the surgical robot according to a type of surgery selected among stored types of surgery.

According to another aspect of the present disclosure, there is provided a method of transporting and placing a surgical robot using a transport robot, the method including transporting the surgical robot by the transport robot, fixing a position of the surgical robot, setting a setup arm of the surgical robot, and mounting a surgical instrument on the surgical robot.

In an embodiment of the present disclosure, the transporting of the surgical robot may include selecting the surgical robot to be transported and the transport robot configured to transport the selected surgical robot.

In an embodiment of the present disclosure, the transporting of the surgical robot may include obtaining input information indicating one of automatic placement and manual placement for the surgical robot.

In an embodiment of the present disclosure, the method may further include obtaining selection information for placement of the surgical robot through a display operation, when the input information indicating the automatic placement is obtained.

In an embodiment of the present disclosure, the method may further include allowing the surgical robot to be moved under user control when the input information indicating the manual placement is obtained.

In an embodiment of the present disclosure, the transporting of the surgical robot may include transporting the surgical robot to a preset position by the transport robot that autonomously travels.

In an embodiment of the present disclosure, the transporting of the surgical robot may include allowing the surgical robot to be moved through manual manipulation when a change in the position of the surgical robot is required after the surgical robot is transported by the transport robot.

In an embodiment of the present disclosure, the fixing of the position of the surgical robot may include operating a fixing part of the surgical robot in response to an approval signal received via a robot display to fix the position of the surgical robot.

In an embodiment of the present disclosure, the setting of the setup arm of the surgical robot may include obtaining input information indicating one of automatic setting and manual setting for a posture of the setup arm.

In an embodiment of the present disclosure, the method may further include obtaining selection information for the posture of the setup arm through a display operation when the input information indicating the automatic setting is obtained.

In an embodiment of the present disclosure, the method may further include obtaining brake release information of the setup arm when the input information indicating the manual setting is obtained.

In an embodiment of the present disclosure, the setting of the setup arm of the surgical robot may include changing the setup arm to a preset posture.

In an embodiment of the present disclosure, in the setting of the setup arm of the surgical robot, when a change in a posture of the setup arm is required after the posture of the setup arm is automatically changed, the posture of the setup arm may be adjustable through manual manipulation.

In an embodiment of the present disclosure, the method may further include resetting a posture of the setup arm or resetting the position of the surgical robot, after the posture of the setup arm of the surgical robot is changed.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a conceptual diagram illustrating a surgical robot system according to an embodiment of the present disclosure;

FIG. 2 is a block diagram illustrating internal configurations of a master robot and a surgical robot of the surgical robot system of FIG. 1;

FIG. 3 is a perspective view illustrating the surgical robot according to an embodiment of the present disclosure;

FIG. 4 is a perspective view illustrating a surgical robot according to another embodiment of the present disclosure;

FIG. 5 is a block diagram illustrating an internal configuration of a transport robot of the surgical robot system shown in FIG. 1;

FIGS. 6A to 6B are views for describing an example in which the transport robot according to an embodiment of the present disclosure is connected to the surgical robot;

FIGS. 7A and 7B are views for describing another example in which the transport robot according to an embodiment of the present disclosure is connected to the surgical robot;

FIGS. 8 and 9 are block diagrams for describing relationships among components of the surgical robot system according to an embodiment of the present disclosure;

FIG. 10 is a flowchart for describing an example of a method of transporting and placing the surgical robot by the transport robot according to an embodiment of the present disclosure;

FIG. 11 is a flowchart illustrating a scenario of the method of transporting and placing the surgical robot performed using the surgical robot system according to an embodiment of the present disclosure; and

FIGS. 12A to 12F are views schematically illustrating a process in which the surgical robot is placed by the transport robot in the surgical robot system according to an embodiment of the present disclosure.

### DETAILED DESCRIPTION

Hereinafter, the following embodiments will be described in detail with reference to the accompanying drawings. When describing with reference to the drawings, identical or corresponding components will be assigned the same reference numerals, and duplicate descriptions thereof will be omitted.

Since various modifications may be made to these embodiments, specific embodiments will be illustrated in the drawings and described in detail. The effects and features of the present embodiments, and methods for achieving the same, will become apparent from the following description taken in conjunction with the accompanying drawings. However, the present embodiments are not limited to the embodiments disclosed below, but may be implemented in various forms.

In describing the present disclosure, a detailed description of related known art will be omitted when it is determined that it may unnecessarily obscure the gist of the present disclosure.

In the following embodiments, singular forms are intended to include plural forms as well, unless the context clearly indicates otherwise. Although terms such as “first,” “second,” and the like may be used to describe various components, such components should not be limited to the above terms. These terms are used only to distinguish one component from another.

In the following embodiments, terms such as “include” or “have” mean that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another member from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

In the following embodiments, an x-axis, a y-axis, and a z-axis are not limited to three axes of the rectangular coordinate system, and may be interpreted in a broader sense. For example, the x-axis, the y-axis, and the z-axis may be perpendicular to one another, or may represent different directions that are not perpendicular to one another.

In cases where certain embodiments may be implemented otherwise, a specific process sequence may be performed differently from the described sequence. For example, two consecutively described processes may be performed substantially at the same time or performed in an order opposite to the described order.

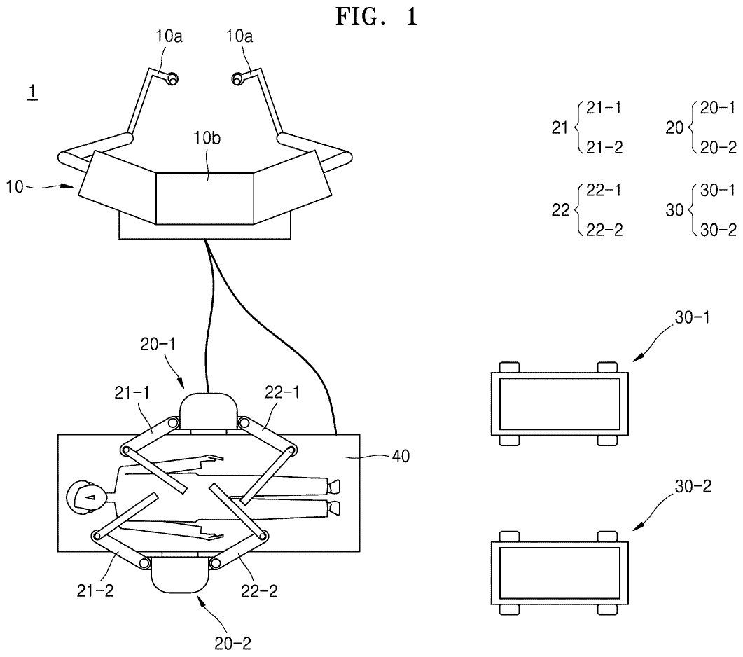

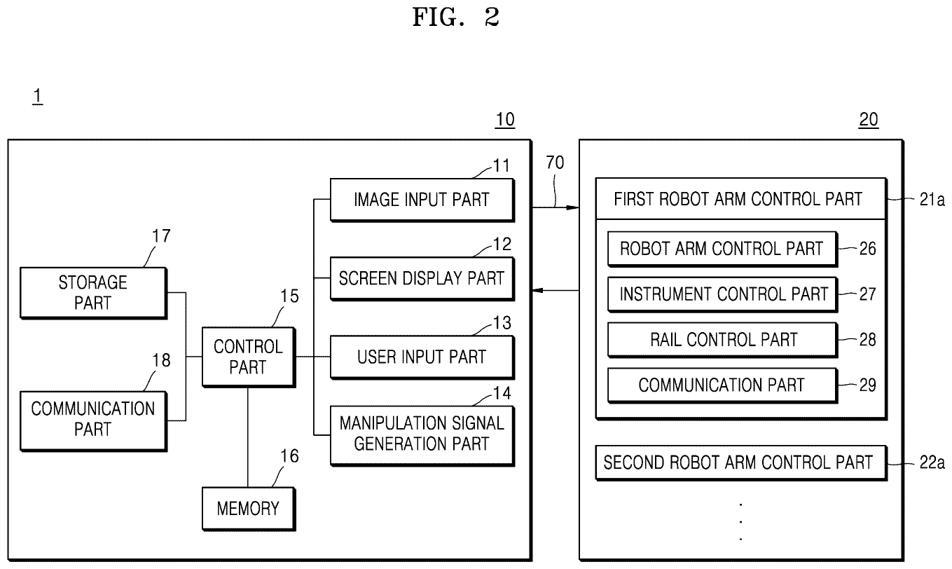

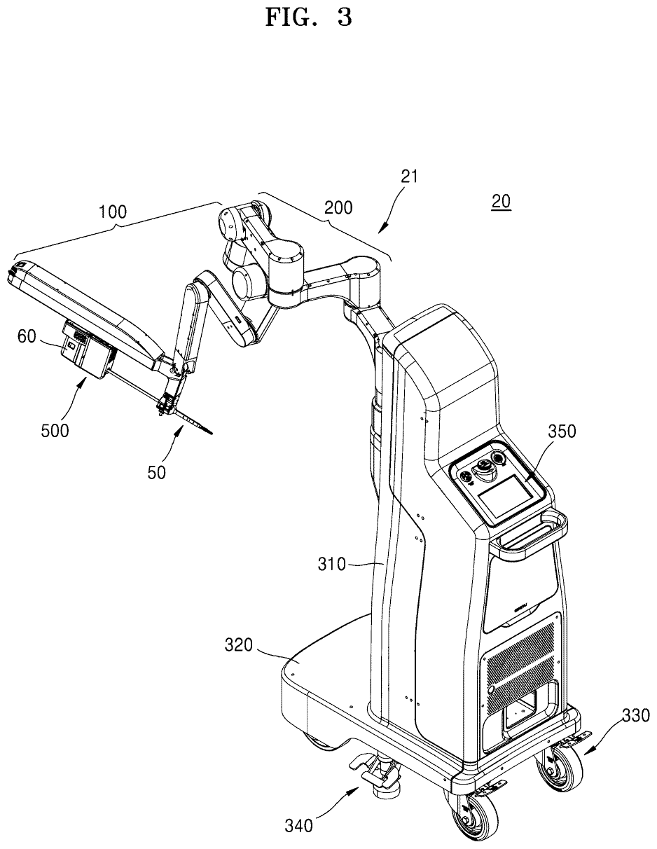

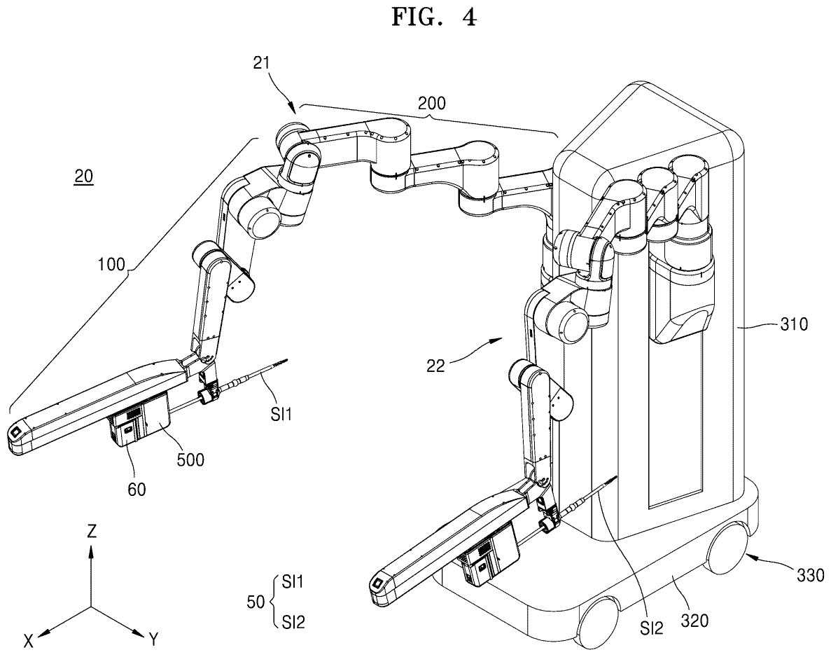

FIG. 1 is a conceptual diagram illustrating a surgical robot system according to an embodiment of the present disclosure, and FIG. 2 is a block diagram illustrating internal configurations of a master robot and a surgical robot of the surgical robot system of FIG. 1. FIG. 3 is a perspective view illustrating the surgical robot according to an embodiment of the present disclosure, and FIG. 4 is a perspective view illustrating a surgical robot according to another embodiment of the present disclosure.

Referring to FIGS. 1 to 4, a surgical robot system 1 includes a master robot 10, a surgical robot 20, a transport robot 30, a surgical bed 40, and a surgical instrument 50.

The master robot 10 includes a manipulation member 10a and a display member 10b, and the surgical robot 20 includes one or more robot arm units 21 and 22.

In detail, the master robot 10 includes the manipulation member 10a that allows a surgical operator to grip and manipulate the manipulation member 10a with both hands. As illustrated in FIG. 1, the manipulation member 10a may be implemented as two or more handles, and manipulation signals generated by operation of the handles may be transmitted to the surgical robot 20 through a wired or wireless communication network to control the robot arm units 21 and 22. That is, surgical motions such as positional movement, rotation, and cutting operations of the robot arm units 21 and 22 may be performed by the surgical operator manipulating the handles.

For example, the surgical operator may manipulate the robot arm units 21 and 22 using manipulation levers in the form of a handle. Such manipulation levers may have various mechanical configurations according to the manipulation method thereof, and may be provided in various configurations for operating the robot arm units 21 and 22 of the surgical robot 20 and/or other surgical instruments, such as a master handle manipulating the motion of each of the robot arm units 21 and 22 and various input tools added to the master robot 10 for manipulating the functions of the entire system such as joystick, keypad, trackball, foot pedal, and touch screen. Here, the manipulation member 10a is not limited to the form of a handle and may have any suitable form as long as it is capable of controlling motions of the robot arm units 21 and 22 through a network such as a wired or wireless communication network.

Alternatively, a voice input or a motion input may also be applied for user input. That is, a user may wear glasses or a head-mounted display (HMD) on the user's head, to which a sensor is attached, and a laparoscope (not shown) included in the surgical robot 20 may move according to a direction in which the user's gaze is directed. Alternatively, when the user issues a voice command such as “left,” “right,” “first arm,” or “second arm,” the voice command may be recognized and a corresponding motion may be performed.

An image captured through the laparoscope to be described later is displayed as a screen image on the display member 10b of the master robot 10. In addition, a predetermined virtual control panel may be displayed separately from, or together with the image captured by the laparoscope on the display member 10b. A detailed description of the arrangement, configuration, and the like of such a virtual control panel will be omitted.

Here, the display member 10b may include one or more monitors, each of which may individually display information necessary for surgery. The number of monitors may be variously determined depending on the type or kind of information that needs to be displayed.

In some embodiments, the surgical robot 20 may include one or more robot arm units 21 and 22. Here, each of the robot arm units 21 and 22 may be provided in the form of a module that can operate independently of each other, and in this case, an algorithm for preventing a collision between the robot arm units 21 and 22 may be applied to the surgical robot system 1.

In general, a robot arm refers to a device having a function similar to that of the arm and/or the wrist of a human being and having a wrist portion to which a predetermined tool may be attached. In the present specification, the robot arm units 21 and 22 may each be defined as a concept encompassing all of the components such as an upper arm, a lower arm, a wrist, and an elbow, a surgical instrument coupled to the wrist portion, and the like. Alternatively, the robot arm unit may also be defined as a concept that includes only components for driving the surgical instrument, excluding the surgical instrument coupled to the wrist portion.

The robot arm units 21 and 22 of the surgical robot 20 described above may be implemented to be driven with multiple degrees of freedom. The robot arm units 21 and 22 may include, for example, a surgical instrument inserted into a surgical site of a patient, a yaw drive part configured to rotate the surgical instrument in a yaw direction according to a surgical position, a pitch drive part configured to rotate the surgical instrument in a pitch direction perpendicular to a rotation direction of the yaw drive part, a transfer drive part configured to move the surgical instrument in a length direction, a rotation drive part configured to rotate the surgical instrument, and a surgical instrument drive part for incising or cutting the surgical lesion by driving an end effector at a distal end of the surgical instrument. However, the configuration of the robot arm units 21 and 22 is not limited thereto, and it should be understood that this example does not limit the scope of the present disclosure. A detailed description of an actual control process in which the robot arm units 21 and 22 rotate and move in response to manipulation of the manipulation member 10a by the surgical operator will be omitted.

The surgical robot system 1 according to an embodiment of the present disclosure may include one or more surgical robots 20. FIG. 1 illustrates an embodiment in which the surgical robot system 1 includes two surgical robots. Here, the two surgical robots may be referred to, for convenience, as a first surgical robot 20-1 and a second surgical robot 20-2. That is, FIG. 1 illustrates an embodiment in which the first surgical robot 20-1 including two robot arm units 21-1 and 22-1 and the second surgical robot 20-2 including two robot arm units 21-2 and 22-2 are arranged, such that a total of four robot arm units 21-1, 22-1, 21-2, and 22-2 are arranged.

In an embodiment, the surgical instrument 50 may be attached to each of two or more of the robot arm units 21-1, 22-1, 21-2, and 22-2, and a laparoscope may be attached to one or more of the robot arm units. In addition, a surgeon may select, via the master robot 10, the robot arm unit 21-1, 22-1, 21-2, or 22-2 to be controlled. As such, by directly manipulating a total of three or more surgical instruments through the master robot 10, the surgeon may accurately and freely perform manipulation of multiple instruments on the surgical bed 40 according to the surgeon's intention, without the need for a surgical assistant.

In some embodiments, one or more surgical robots 20 may be provided to perform surgery on a patient, and the laparoscope for allowing a surgical site to be displayed as a screen image through the display member 10b may be implemented as an independent surgical robot 20.

In addition, as described above, the embodiments of the present disclosure may be widely applicable to surgeries employing various surgical endoscopes other than a laparoscope (e.g., a thoracoscope, an arthroscope, a rhinoscope, and the like).

Referring to FIG. 2, in an embodiment of the present disclosure, the master robot 10 may include an image input part 11, a screen display part 12, a user input part 13, a manipulation signal generation part 14, a control part 15, a memory 16, a storage part 17, and a communication part 18.

The image input part 11 may receive an image captured by a camera provided in the laparoscope of the surgical robot 20 through a wired or wireless communication network.

The screen display part 12 outputs a screen image corresponding to the image received through the image input part 11 as visual information. In addition, the screen display part 12 may further output information corresponding to biometric information of a subject to be treated, when the biometric information is input. In addition, the screen display part 12 may further output patient image data related to a surgical site (e.g., an X-ray image, a computed tomography (CT) image, a magnetic resonance imaging (MRI) image, or the like). Here, the screen display part 12 may be implemented in the form of a display member (see 10b of FIG. 1), and an image processing process for allowing the received image to be output as a screen image through the screen display part 12 may be performed by the control part 15.

In the embodiment illustrated in FIG. 2, the image input part 11 and the screen display part 12 are illustrated as being included in the master robot 10, but the present disclosure is not limited thereto. That is, the display member may be provided as a separate member spaced apart from the master robot 10. Alternatively, the display member may also be provided as one component of the master robot 10. In addition, in another embodiment, a plurality of display members may be provided, one of which may be positioned adjacent to the master robot 10, and others thereof may be positioned at some distance from the master robot 10.

Here, the screen display part 12 (that is, the display member 10b of FIG. 1) may be provided as a three-dimensional display device. In detail, the three-dimensional display device refers to an image display device that adds depth information to a two-dimensional image by applying a stereoscopic technique and uses the depth information to enable an observer to perceive a three-dimensional effect and a sense of presence. The surgical robot system 1 according to an embodiment of the present disclosure may provide a user with a more realistic virtual environment by including a three-dimensional display device as the screen display part 12.

The user input part 13 is a member that allows a surgical operator to control movement and operation functions of the transport robot 30 or to control positions and functions of robot arm units 21 and 22 of the surgical robot 20. The user input part 13 may be implemented as a handle-shaped manipulation member (see the manipulation member 10a in FIG. 1) as illustrated in FIG. 1. However, the shape thereof is not limited thereto and may be implemented in various shapes to achieve the same purpose. In addition, for example, a portion of the user input part 13 may be configured as a handle, and another portion thereof may be configured in a different form, such as a clutch button. In addition, a finger guide, such as a finger insertion tube or a finger ring, may be further provided to allow a surgical operator's finger to be inserted and secured, thereby facilitating manipulation of a surgical tool.

When a surgical operator operates the user input part 13 to manipulate positional movement or a surgical operation of the robot arm units 21 and 22, or when the surgical operator operates the user input part 13 to manipulate movement or an operation of the transport robot 30, the manipulation signal generation part 14 may generate a corresponding manipulation signal.

For example, the manipulation signal generation part 14 may transmit the generated manipulation signal to the control part 15, or may transmit the generated manipulation signal to the surgical robot 20 through the communication part 18, and may also transmit the generated manipulation signal to the transport robot 30. The manipulation signal may be transmitted and received via a wired or wireless communication network. Based on the transmitted manipulation signal, the control part 15 may control the surgical robot 20 or the transport robot 30 to operate. Alternatively, based on the transmitted manipulation signal, a robot arm control part 26 included in the surgical robot 20 may control the robot arm units 21 and 22 to operate. Alternatively, based on the transmitted manipulation signal, an instrument control part 27 included in the surgical robot 20 may control the surgical instrument 50 to operate. However, the method of controlling the operation of the surgical robot 20 or the transport robot 30 based on the manipulation signal is not limited to the above-described examples.

The control part 15 may include at least one processor and may control operations of each component so that the above-described functions can be performed. In an example, the control part 15 may perform a function of converting an image input through the image input part 11 into a screen image to be displayed through the screen display part 12.

The memory 16 may perform a function of temporarily or permanently storing data processed by the control part 15. Here, the memory 16 may include a magnetic storage medium or a flash storage medium, but the scope of the present disclosure is not limited thereto.

The storage part 17 may store data received from the surgical robot 20. In addition, the storage part 17 may store various pieces of input data (e.g., patient data, device data, surgery data, and the like).

The communication part 18 interfaces with a communication network 70 to provide a communication interface necessary for transmitting and receiving image data transmitted from the surgical robot 20 and control data transmitted from the master robot 10. In addition, the communication part 18 provides an interface required to transmit and receive data transmitted from the transport robot 30 and control data transmitted from the master robot 10 to the transport robot 30 in conjunction with the communication network 70.

The surgical robot 20 includes a plurality of robot arm unit control parts 21a and 22a. In addition, the robot arm unit control part 21a includes the robot arm control part 26, the instrument control part 27, and a communication part 29. In addition, the robot arm unit control part 21a may further include a rail control part 28.

Referring to FIG. 2, the rail control part 28 may control a movement path such that the surgical instrument 50 in the robot arm units 21 and 22 is movable along a preset path. The robot arm control part 26 may receive a manipulation signal generated by the manipulation signal generation part 14 of the master robot 10, and may serve to control the robot arm units 21 and 22 so as to operate according to the manipulation signal. The instrument control part 27 may receive a manipulation signal generated by the manipulation signal generation part 14 of the master robot 10, and may serve to control the surgical instrument 50 so as to operate according to the manipulation signal. The communication part 29 interfaces with the communication network 70 to provide a communication interface necessary for transmitting and receiving image data transmitted from the surgical robot 20 and control data transmitted from the master robot 10.

In some embodiments, the communication network 70 may serve to connect the master robot 10 to the surgical robot 20. That is, the communication network 70 refers to a communication network for providing an access path so that data can be transmitted and received between the master robot 10 and the surgical robot 20 after the master robot 10 is connected to the surgical robot 20. The communication network 70 may be, for example, a wired network such as local area networks (LANs), wide area networks (WANs), metropolitan area networks (MANs), and integrated services digital networks (ISDNs), or a wireless network such as wireless LANs, code division multiple access (CDMA), Bluetooth, and satellite communication, but the scope of the present disclosure is not limited thereto.

Similarly, although not illustrated in the drawings, the communication network 70 may serve to connect the master robot 10 to the transport robot 30. That is, the communication network 70 may provide an access path so that data can be transmitted and received between the master robot 10 and the transport robot 30 after the master robot 10 is connected to the transport robot 30.

Referring to FIGS. 3 and 4, the surgical robot 20 applied to the surgical robot system 1 according to an embodiment of the present disclosure may include a first main body 310, a second main body 320, the robot arm unit, a display part 350, wheels 330, fixing parts 340, and the like.

FIG. 3 illustrates an embodiment in which the surgical robot 20 includes one robot arm unit, and FIG. 4 illustrates an embodiment in which the surgical robot 20 includes two robot arm units. In the embodiment illustrated in FIG. 4, the robot arm units are defined as a first robot arm unit 21 and a second robot arm unit 22, respectively.

The first main body 310 functions as a body of the surgical robot 20, and the first robot arm unit 21 and the second robot arm unit 22 may be disposed on the first main body 310. In addition, the first main body 310 may provide a reference point for driving of the first robot arm unit 21 and the second robot arm unit 22.

The second main body 320 may support the first main body 310. In addition, the second main body 320 may include the wheels 330 and the fixing parts 340. In the embodiment illustrated in FIG. 3, the second main body 320 includes four wheels 330 and two fixing parts 340, but the shapes and numbers of the wheels 330 and the fixing part 340 are not limited thereto.

Here, the wheels 330 may be provided as non-driven wheels that are not connected to a driving source. That is, when the surgical robot 20 is moved by the transport robot 30 or is moved manually, the wheels 330 of the surgical robot 20 may serve as auxiliary movement members.

In some embodiments, the fixing parts 340 provided on the second main body 320 may serve to fix a position of the surgical robot 20 such that the surgical robot 20 is fixed in place after being placed. Accordingly, after the position of the surgical robot 20 is fixed by the fixing parts 340, it is possible to prevent the surgical robot 20 from being moved by vibration generated by operation of the surgical robot 20 or by external forces.

The first robot arm unit 21 is disposed on one side of the first main body 310, and a first surgical instrument SI1 may be mounted thereon. The surgical robot 20 may drive the first robot arm unit 21 to adjust a position and a posture of the first surgical instrument SI1.

The first robot arm unit 21 may have a plurality of extension links, and a posture of the first robot arm unit 21 may be determined according to driving of each of the plurality of extension links.

The second robot arm unit 22 is disposed on another side of the first main body 310, and a second surgical instrument SI2 may be mounted thereon. The surgical robot 20 may drive the second robot arm unit 22 to adjust a position and a posture of the second surgical instrument SI2.

The second robot arm unit 22 may have a plurality of extension links, and a posture of the second robot arm unit 22 may be determined according to driving of each of the plurality of extension links.

A motor pack 500 is connectable to the surgical instrument 50, and may be coupled to the surgical robot 20, specifically to a robot arm unit, and may be fixed in position.

An instrument case 60 is connected to one side of the surgical instrument 50, and the motor pack 500 is connected and coupled to another side opposite thereto. The motor pack 500 receives power from an external source to generate driving power, and may transmit the driving power generated by the motor pack 500 to the surgical instrument 50. Accordingly, the surgical instrument 50 may perform a pitch motion, a yaw motion, an actuation motion, and a roll motion.

The display part 350 may provide a virtual control panel configured to directly manipulate the surgical robot 20. In addition, the virtual control panel may be displayed together with an image captured through the laparoscope or the like, or may be displayed independently.

In an embodiment, the robot arm unit of the surgical robot 20 will be described in detail.

The robot arm unit may include a setup arm 200 and an active arm 100. The setup arm 200 may be positioned outside the surgical bed 40 on which a patient is placed, and may be connected to the active arm 100.

The setup arm 200 serves to set a position and a posture of the active arm 100 such that the surgical instrument 50 coupled to the active arm 100 can be disposed at a position suitable for surgery. The setup arm 200 operates only in a pre-surgery stage to set up a position, and once the surgery starts, the setup arm 200 is fixed in position while remaining stationary.

That is, the setup arm 200 may be configured to be operable only during a setup period during which the surgical robot arm is positioned on one side of the patient. The setup arm 200 may be manually operated by a user such as medical staff to change a position thereof.

The setup arm 200 according to an embodiment of the present disclosure may include a setup link assembly having a plurality of setup links.

In an optional embodiment, the setup arm 200 may include a drive part (not shown), and various modifications are possible such that a user may change a position of the setup arm 200 using an external device such as a controller (not shown).

In some embodiments, the active arm 100 may be a portion that moves in real time by manipulation of a surgeon during surgery. Specifically, the active arm 100 is coupled to an end portion of the setup arm 200, which is fixed in a non-movable state after a position thereof is adjusted in a pre-surgery stage, and may be a portion that moves in real time by manipulation of the surgeon during surgery. The active arm 100 may be rotatably and movably connected to one side of the setup arm 200, and the surgical instrument 50 and a trocar (not shown) may be coupled thereto.

The surgical robot arm according to an embodiment of the present disclosure sets a virtual pivot center at a predetermined position of a distal end of the surgical instrument 50 mounted on the active arm 100 coupled to one end portion of the setup arm 200 fixed in position, and controls the active arm 100 such that the surgical instrument 50 rotates about the virtual pivot center. The virtual pivot center is a remote center of motion (RCM). That is, the surgical instrument 50 may rotate around the RCM point to perform a yaw motion and a pitch motion.

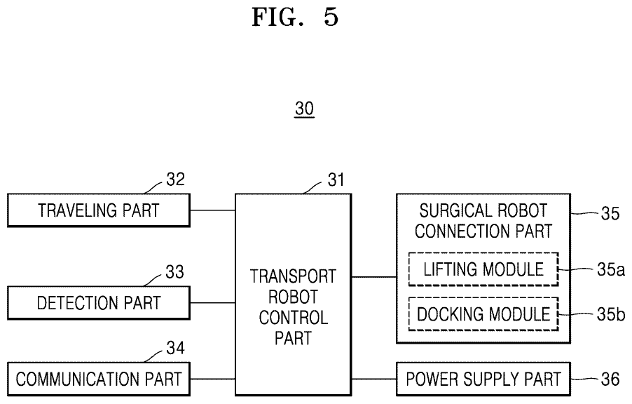

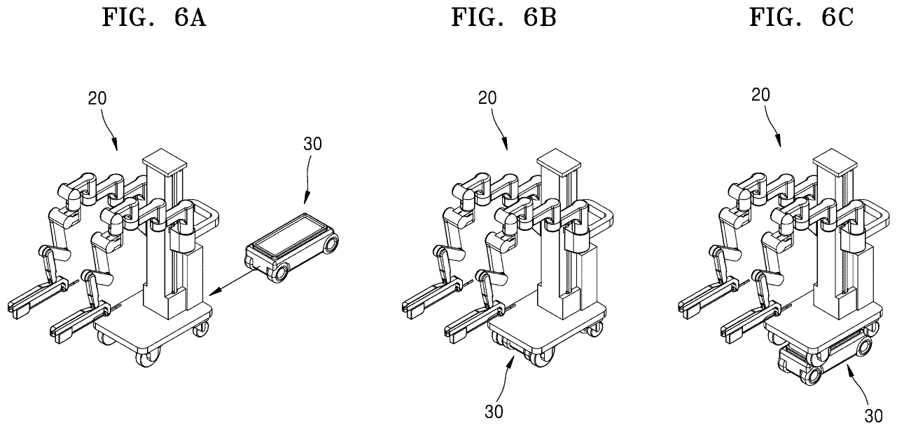

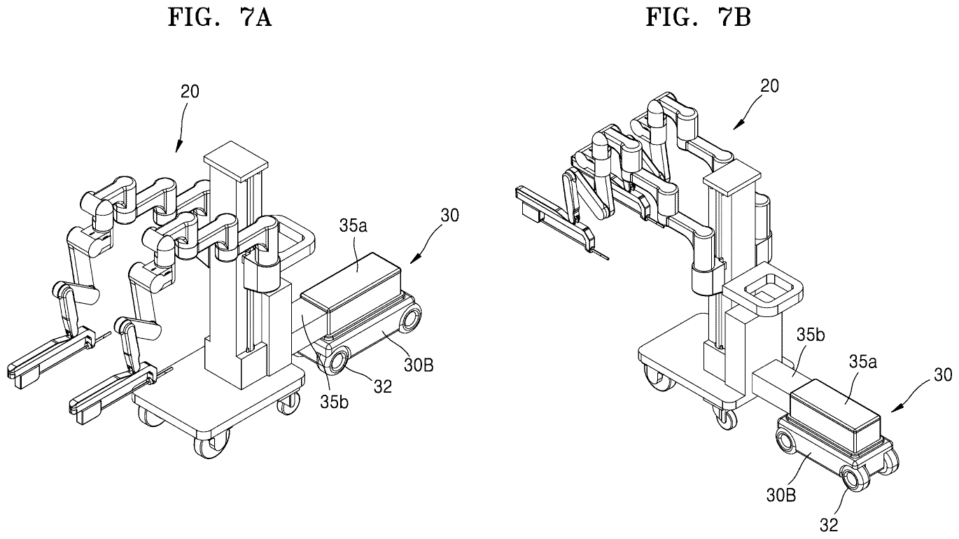

FIG. 5 is a block diagram illustrating an internal configuration of the transport robot 30 of the surgical robot system 1 shown in FIG. 1. FIGS. 6A to 6C are views for describing an example in which the transport robot 30 according to an embodiment of the present disclosure is connected to the surgical robot 20, and FIGS. 7A and 7B are views for describing another example in which the transport robot 30 according to an embodiment of the present disclosure is connected to the surgical robot 20.

Referring to FIGS. 5 to 7B, the transport robot 30 according to an embodiment of the present disclosure may include a transport robot control part 31, a traveling part 32, a detection part 33, a communication part 34, a surgical robot connection part 35, a power supply part 36, and a body 30B in which these components are disposed.

In defining each portion of the body 30B of the transport robot 30, a portion of the body 30B facing a ceiling within a traveling area is defined as an upper surface portion, and a portion of the body 30B facing a floor within the traveling area is defined as a lower surface portion. In addition, among portions forming a periphery of the body 30B between the upper surface portion and the lower surface portion, a portion facing a traveling direction is defined as a front surface portion, and a portion facing a direction opposite to the front surface portion is defined as a rear surface portion.

The traveling part 32 may be a member for performing a role of moving or stopping the body 30B. Specifically, the traveling part 32 may include a driving source, wheels, and the like, which are involved in movement, steering, and stopping of the body 30B. The driving source may be an electric motor, but is not necessarily limited thereto. The wheels may include one or more driving wheels that receive driving force from the driving source, and non-driven wheels that do not receive driving force and rotate in accordance with movement of the transport robot 30. When a plurality of driving wheels and a plurality of driving sources are provided, the plurality of driving sources may be respectively connected to different driving wheels, and rotation of each driving wheel may be independently controlled.

Although driving wheels having a wheel shape are illustrated in the drawings, the concept of the present disclosure is not limited thereto, and the traveling part 32 may employ an endless track or another type of moving member.

The surgical robot connection part 35 may be a member configured to be brought into contact with and connected to the surgical robot 20. That is, the surgical robot connection part 35 may serve as a member by which the transport robot 30 is physically connectable to the surgical robot 20, and may be formed such that the transport robot 30 is able to transport the surgical robot 20.

In an embodiment, the surgical robot connection part 35 may include a lifting module 35a and/or a docking module 35b.

Here, the lifting module 35a is a device capable of raising and lowering the surgical robot 20. For example, the transport robot 30 may lift the surgical robot 20 from a traveling surface by using the lifting module 35a.

As illustrated in FIGS. 6A to 6C, the transport robot 30 may enter below the second main body 320 of the surgical robot 20 and lift the surgical robot 20 through the lifting module 35a. In addition, the transport robot 30 may transport the surgical robot 20 while the surgical robot 20 is in a lifted state.

The lifting module 35a may be accommodated in the body 30B of the transport robot 30, and may protrude outward from the body 30B when necessary. For example, the lifting module 35a may be raised by a hydraulic method.

An upper surface of the lifting module 35a may include a structure to be fastened to the second main body 320 of the surgical robot 20. Accordingly, when the lifting module 35a is raised in a state in which the transport robot 30 has moved below the second main body 320 of the surgical robot 20, the upper surface of the lifting module 35a and a lower surface of the second main body 320 may be fastened to each other. Accordingly, while the transport robot 30 transports the surgical robot 20, the transport robot 30 may stably transport the surgical robot 20 while being coupled to the surgical robot 20.

However, the lifting module 35a and the surgical robot 20 may be coupled by a simple coupling structure, such that, when the lifting module 35a is lowered again, the lifting module 35a may be easily detached from the second main body 320.

In some embodiments, the docking module 35b is a device capable of being connected to the first main body 310 of the surgical robot 20. For example, the docking module 35b may extend from the front surface portion of the body 30B of the transport robot 30 or may extend from the rear surface portion of the body 30B, and may be connected to the first main body 310 of the surgical robot 20. Alternatively, as in the embodiment illustrated in FIGS. 7A and 7B, the docking module 35b may be disposed to extend from the lifting module 35a.

That is, while the transport robot 30 is connected to the surgical robot 20 via the docking module 35b, the transport robot 30 may pull the surgical robot 20 or push the surgical robot 20 from behind to move the surgical robot 20.

Specifically, referring to FIGS. 7A and 7B, when the docking module 35b is disposed on the front surface portion of the transport robot 30, the docking module 35b may be connected to a rear surface portion of the surgical robot 20, such that the transport robot 30 may move the surgical robot 20 by pushing the surgical robot 20.

Alternatively, when the docking module 35b is disposed on the rear surface portion of the transport robot 30, the docking module 35b may be connected to a front surface portion of the surgical robot 20, such that the transport robot 30 may move the surgical robot 20 by pulling the surgical robot 20.

However, the concept of the present disclosure is not limited thereto, and the transport robot 30 may be connected to the surgical robot 20 via the docking module 35b while being positioned at a side surface of the surgical robot 20, and may move the surgical robot 20 while being positioned alongside the surgical robot 20.

The detection part 33 may detect a surrounding environment of the transport robot 30 and may assist traveling of the transport robot 30. The detection part 33 may detect an external object located in a surgical space in which the transport robot 30 travels. Specifically, the detection part 33 may detect a direction toward an external object and a separation distance between the transport robot 30 and the external object.

For example, the detection part 33 may detect a position of the surgical bed 40 and a position of the surgical robot 20. In addition, when a plurality of transport robots 30 are provided, the detection part 33 of one transport robot 30 may detect a position of another transport robot 30.

The detection part 33 may include at least one of a camera sensor and an optical sensor. Examples thereof may include a two-dimensional laser scanner (LiDAR), a three-dimensional vision (stereo) camera, a proximity sensor, and the like.

The communication part 34 may perform communication with other components of the surgical robot system 1, such as the master robot 10 and the surgical robot 20. The communication part 34 may support wireless communication methods such as Bluetooth, Wi-Fi, ultra-wideband (UWB), near-field communication (NFC), and mobile communication networks (4G and 5G), and may be connected to an external device to transmit and receive data, but the scope of the present disclosure is not limited thereto.

The power supply part 36 is a member for supplying power to the surgical robot 20. The transport robot 30 according to an embodiment of the present disclosure may include a battery built thereinto, and the built-in battery may be connected to an external device to supply power to the external device. That is, the transport robot 30 includes a built-in battery and may provide power to the surgical robot 20 when necessary.

In some embodiments, the transport robot 30 may provide power to the surgical robot 20 through the power supply part 36 in a wired or wireless manner. For example, the transport robot 30 may supply power to the surgical robot 20 using USB power delivery (USB-PD) or wireless charging technology.

In an embodiment, power supply may be performed even without raising and lowering the surgical robot 20 through the lifting module 35a, and may be performed via a power cable or via a direct docking connection between the surgical robot 20 and the transport robot 30. For example, the power supply part 36 may be disposed together with the above-described docking module 35b. As illustrated in FIGS. 7A and 7B, when the transport robot 30 is connected to the surgical robot 20 via the docking module 35b, the power supply part 36 may also be connected to the surgical robot 20. Accordingly, when an emergency situation occurs, such as a situation in which power supply in an operating room is cut off, the transport robot 30 may supply power to the surgical robot 20 using the built-in battery held by the transport robot 30.

In an embodiment of the present disclosure, the transport robot 30 may be capable of autonomous traveling within a preset area. For example, the transport robot 30 may autonomously travel within an operating room area in which the surgical bed 40 is disposed, in order to approach the surgical robot 20 and transport the surgical robot 20.

In some embodiments, the transport robot 30 may be an autonomous mobile robot (AMR). That is, the transport robot 30 may be capable of flexible movement through real-time perception of the environment and autonomous path-planning functions. The transport robot 30 may scan a surrounding environment in real time through the detection part 33, and may generate and update a map and estimate a current position based thereon. Accordingly, the transport robot 30 may recognize an environment and autonomously determine a traveling path even without a predefined path.

The transport robot 30 may set a traveling path using a global path planning technique, which is a function of setting an optimal path to a destination, and a local path planning technique, which is a function of adjusting the path while avoiding obstacles detected during movement in real time.

Through this, the transport robot 30 may safely operate even in a space in which people move, and may promptly respond to unpredictable changes in an environment.

The surgical robot system 1 according to an embodiment of the present disclosure may transport the surgical robot 20 by utilizing the above-described autonomous traveling functions.

In an embodiment, the transport robot 30 may move toward the surgical robot 20 according to a received signal, may be connected to the surgical robot 20, and may place the surgical robot 20 at a preset position.

For example, the transport robot 30 may receive a manipulation signal generated by manipulation of the master robot 10 and may move toward the selected surgical robot 20. In addition, the transport robot 30 may be connected to the surgical robot 20 via the above-described surgical robot connection part 35. The transport robot 30 connected to the surgical robot 20 may autonomously travel to a position at which the surgical robot 20 is to be placed and may transport the surgical robot 20. After the surgical robot 20 is transported to an appropriate position, the transport robot 30 may be detached from the surgical robot 20 automatically or after a user approval procedure. For example, the transport robot 30 may place the surgical robot 20 on a floor by lowering the lifting module 35a.

Here, information regarding a preset position at which the surgical robot 20 is placed may be stored while being classified according to a type of surgery. In addition, the surgical robot 20 may be selected according to a type of surgery selected by a user among stored types of surgery, and the surgical robot 20 that needs to be moved may be automatically moved by the transport robot 30.

That is, the transport robot 30 may place the surgical robot 20 in accordance with the selected type of surgery among the stored types of surgery. This will be described in detail later.

The surgical robot system 1 according to an embodiment of the present disclosure may have an autonomous traveling mode and a travel assist mode among selectable traveling modes for the transport robot 30.

In other words, the surgical robot system 1 may have the autonomous traveling mode in which the transport robot 30 is capable of autonomous traveling and the travel assist mode capable of assisting movement of the surgical robot 20. Such traveling modes may be selected and executed collectively through the master robot 10, or may be selected and executed through individual manipulation of the surgical robot 20 or the transport robot 30.

For example, when the autonomous traveling mode is selected, the transport robot 30 may perform an autonomous traveling function to move toward the surgical robot 20, may be connected to the surgical robot 20, and may transport the surgical robot 20 to a preset position.

In some embodiments, when the travel assist mode is selected, the traveling part 32 of the transport robot 30 may operate under certain conditions. In an embodiment, the transport robot 30 may operate the traveling part 32 by detecting a force transmitted from outside.

In some embodiments, the travel assist mode may be performed while the transport robot 30 is connected to the surgical robot 20. For example, when the transport robot 30 enters below the second main body 320 of the surgical robot 20 and the surgical robot 20 is lifted, a traveling mode of the transport robot 30 may be selected as the travel assist mode.

When the travel assist mode is selected while the transport robot 30 is connected to the surgical robot 20, the transport robot 30 may detect a direction and a force applied to move the surgical robot 20 and may drive the traveling part 32 based on the detected direction and force. In this manner, the transport robot 30 may facilitate movement of the surgical robot 20 by driving the traveling part 32. Specifically, the travel assist mode may be utilized when a user intends to move the surgical robot 20 by manual manipulation. Since the transport robot 30 connected to the surgical robot 20 allows the traveling part 32 to operate in response to an external force, the user may move the surgical robot 20 in a desired direction by merely holding a handle of the surgical robot 20 and pushing the surgical robot 20 in an intended movement direction. Accordingly, the user may manually move the surgical robot 20 with reduced force.

Meanwhile, the surgical bed 40 of the surgical robot system 1 according to an embodiment of the present disclosure may include a sensor configured to transmit position information of the surgical bed 40 to another robot.

In an embodiment, the surgical bed 40 may transmit and receive data to and from the transport robot 30 through a network, in the same manner as communication among the above-described master robot 10, surgical robot 20, and transport robot 30. For example, the transport robot 30 may obtain information on a distance to the surgical bed 40 and a direction toward the surgical bed 40 via a sensor of the surgical bed 40. Since a position at which the surgical robot 20 is to be placed is determined based on the surgical bed 40, the surgical robot 20 may be transported to a proper position based on the position information of the surgical bed 40.

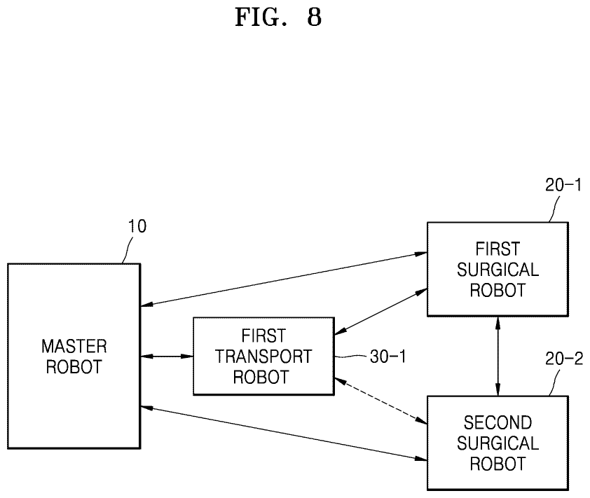

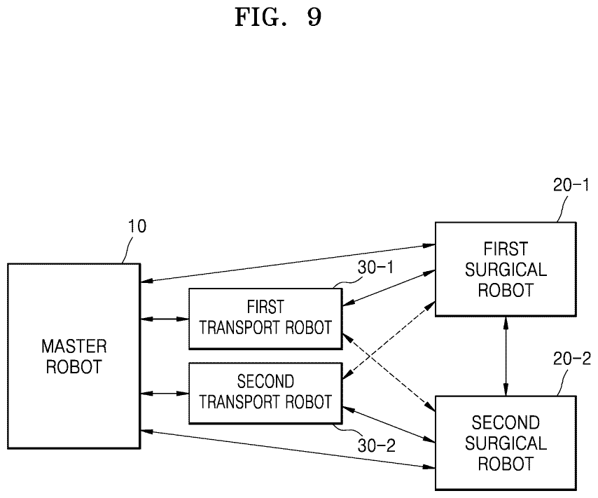

FIGS. 8 and 9 are block diagrams for describing relationships among components of the surgical robot system 1 according to an embodiment of the present disclosure.

FIG. 8 illustrates a case in which the surgical robot system 1 includes the first surgical robot 20-1, the second surgical robot 20-2, and a first transport robot 30-1, and FIG. 9 illustrates a case in which the surgical robot system 1 includes the first surgical robot 20-1, the second surgical robot 20-2, a first transport robot 30-1, and a second transport robot 30-2.

As described above, the master robot 10 may transmit and receive data to and from the surgical robot 20 and the transport robot 30 through a network such as a wired or wireless communication network. In the embodiment illustrated in FIG. 8, the master robot 10 communicates with the first surgical robot 20-1 and the second surgical robot 20-2, and may control each of the first surgical robot 20-1 and the second surgical robot 20-2. Hereinafter, the data transmitted and received among the master robot 10, the surgical robot 20, and the transport robot 30 are merely exemplary, and the types of data transmitted and received among them are not limited thereto.

The first surgical robot 20-1 and the second surgical robot 20-2 may be connected to the master robot 10 through a network, may receive data related to control commands from the master robot 10, and may transmit obtained information, such as image data, to the master robot 10. In addition, the first surgical robot 20-1 and the second surgical robot 20-2 may transmit and receive data, such as movement information and position information, to and from each other.

The first transport robot 30-1 may also be connected to the master robot 10 through a network, may receive data related to control commands from the master robot 10, and may transmit surrounding information obtained through the detection part 33 during movement to the master robot 10. The first transport robot 30-1 may transmit and receive data to and from the first surgical robot 20-1 and the second surgical robot 20-2. In an embodiment, when the transport robot 30 is matched with one surgical robot 20 to be transported among a plurality of surgical robots 20 based on a manipulation signal from the master robot 10, the transport robot 30 may communicate with the corresponding surgical robot 20. For example, the transport robot 30 may first be matched with the first surgical robot 20-1 and transport the first surgical robot 20-1 to a designated position. After placement of the first surgical robot 20-1 is completed, the transport robot 30 may then be matched with the second surgical robot 20-2 to transport the second surgical robot 20-2.

In the embodiment illustrated in FIG. 9, a case is shown in which the first transport robot 30-1 is matched with the first surgical robot 20-1 and the second transport robot 30-2 is matched with the second surgical robot 20-2. In this case, each transport robot 30 communicates with the matched surgical robot 20 and may transport each surgical robot 20 to a designated position.

Hereinafter, a method of transporting and placing the surgical robot 20 through the transport robot 30 in the surgical robot system 1 according to an embodiment of the present disclosure will be described.

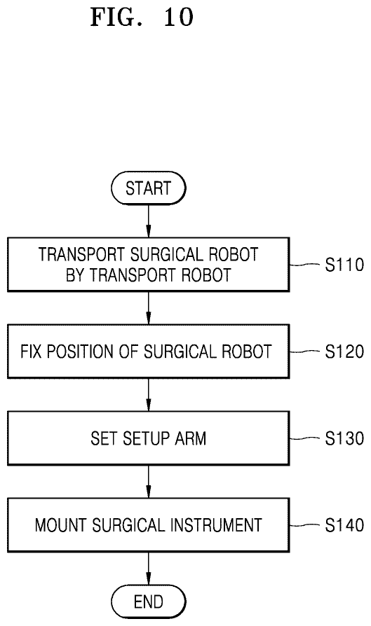

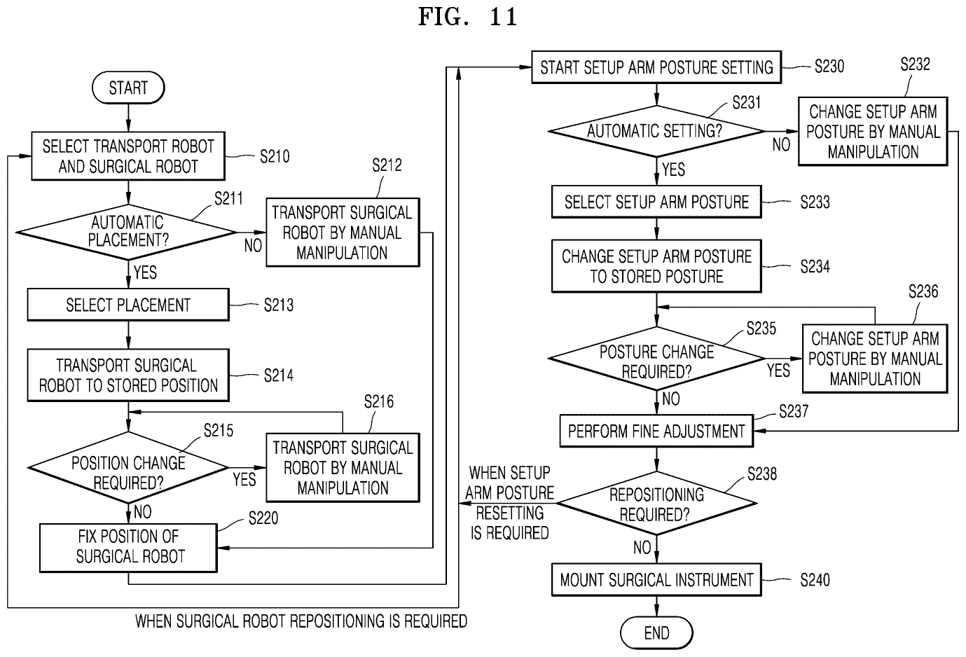

FIG. 10 is a flowchart for describing an example of the method of transporting and placing the surgical robot 20 by the transport robot 30 according to an embodiment of the present disclosure.

Referring to FIG. 10, the method of transporting and placing the surgical robot 20 by the transport robot 30 may include operations S110 to S140. In the surgical robot system 1 according to an embodiment of the present disclosure, the method of transporting and placing the surgical robot 20 by the transport robot 30 includes operations that are performed sequentially over time by the master robot 10, the surgical robot 20, and the transport robot 30 shown in FIGS. 2 and 5 and the like. Accordingly, even when certain descriptions are omitted hereinafter, the descriptions provided above with respect to the apparatuses or the control part 15 illustrated in FIGS. 2 and 5 and the like may also be applied to the method of transporting and placing the surgical robot 20 by the transport robot 30 of FIG. 10.

As described above with reference to FIGS. 2 and 5, at least one of the operations of the method of transporting and placing the surgical robot 20 by the transport robot 30 of FIG. 10 may be processed by the master robot 10, the surgical robot 20, or the transport robot 30.

First, in operation S110, the surgical robot 20 may be transported by the transport robot 30. The transport robot 30 may move toward the surgical robot 20 in response to a received signal, may be connected to the surgical robot 20, and may place the surgical robot 20 at a preset position. An operation signal for transporting the surgical robot 20 may be transmitted to the transport robot 30 by manipulation of the master robot 10 or the surgical robot 20, and the transport robot 30 may move toward the selected surgical robot 20 and transport the surgical robot 20. In this case, the transport robot 30 may transport the surgical robot 20 to a position corresponding to a pre-stored position of the surgical robot 20. Here, the pre-stored position of the surgical robot 20 may be a position at which the surgical robot 20 is to be placed according to a type of surgery.

The transport robot 30 that has approached the surgical robot 20 may be connected to the surgical robot 20. For example, as described above, the transport robot 30 that has entered below the surgical robot 20 may lift the surgical robot 20 through the lifting module 35a. When the surgical robot 20 is lifted by the transport robot 30, the transport robot 30 may load the surgical robot 20 and move the surgical robot 20 to the preset position. After the transport robot 30 is moved to the preset position, the transport robot 30 may lower the lifting module 35a to place the surgical robot 20. Such a placement process of the surgical robot 20 will be described in detail later.

Thereafter, in operation S120, the position of the surgical robot 20 may be fixed. In an embodiment, fixing of the position of the surgical robot 20 may be performed by the fixing parts 340 provided on the second main body 320 of the surgical robot 20. In addition, a fixing device may be provided on the wheel 330 itself of the surgical robot 20, such that operation of the wheel 330 is restricted by manipulation of the fixing device.

In operation S130, the setup arm 200 of the surgical robot 20 may be set. As described above, the setup arm 200 is a portion connected to the active arm 100, and the setup arm 200 may be positioned outside the surgical bed 40 on which a patient is placed.

Here, the setup arm 200 may serve to set a position and a posture of the active arm 100. In other words, the setup arm 200 serves to set the position and the posture of the active arm 100 such that the surgical instrument 50 may be positioned at a position suitable for surgery.

Accordingly, the setup arm 200 may be manipulated before surgery to position the surgical instrument 50 relative to a surgical site of a patient, and may remain stationary during an actual surgery. In contrast, the active arm 100 may be a portion that moves in real time by manipulation of a surgeon during surgery.

In operation S140, a surgical instrument may be mounted on the surgical robot 20. That is, after a posture of the setup arm 200 is set, the surgical instrument 50 may be mounted on the active arm 100. Specifically, the instrument case 60 may be connected to one side of the surgical instrument, and the motor pack 500 may be connected to and coupled to another side opposite thereto. The motor pack 500 receives power from an external source to generate driving power, and may transmit the driving power generated by the motor pack 500 to the surgical instrument 50. Accordingly, the surgical instrument 50 may perform a pitch motion, a yaw motion, an actuation motion, and a roll motion.

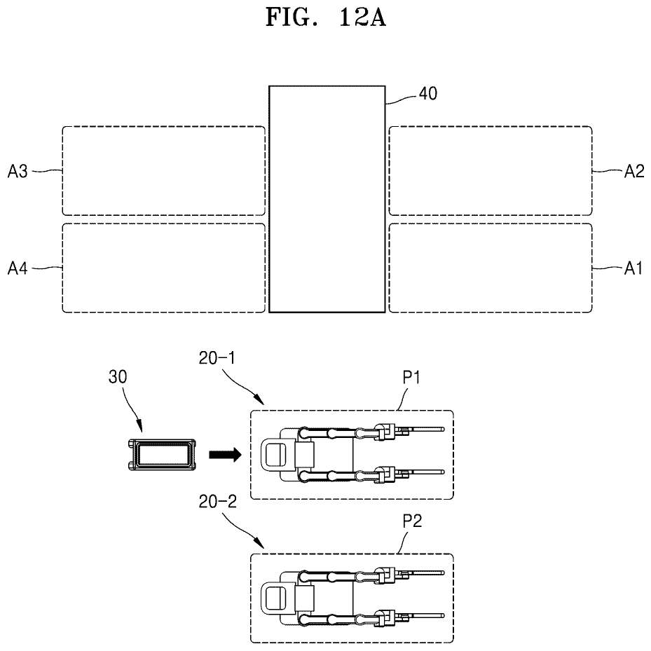

FIG. 11 is a flowchart illustrating a scenario of a method of transporting and placing the surgical robot 20 performed using the surgical robot system 1 according to an embodiment of the present disclosure.

Referring to FIG. 11, the method of transporting and placing the surgical robot 20 according to an embodiment may include operations S210 to S290. In this case, some operations may be substantially the same as the operations of the method of transporting and placing the surgical robot 20 described with reference to FIG. 10. For example, operations S210, S220, S230, and S240 may respectively correspond to operations S110, S120, S130, and S140 described above.

Accordingly, detailed descriptions of contents overlapping with operations S110 to S140 described above will be omitted, and hereinafter, additional or modified technical features will be mainly described.

In operation S210, when a plurality of surgical robots 20 or a plurality of transport robots 30 are provided, the surgical robot 20 to be transported and the transport robot 30 to transport the surgical robot 20 may be selected.

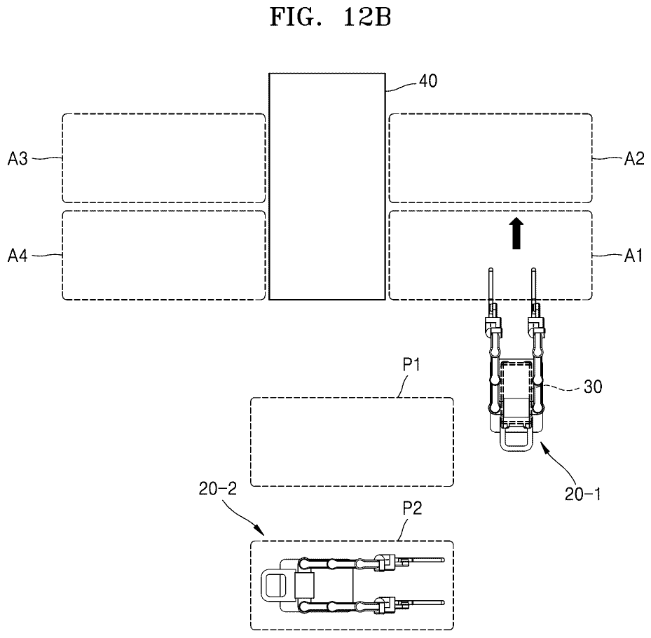

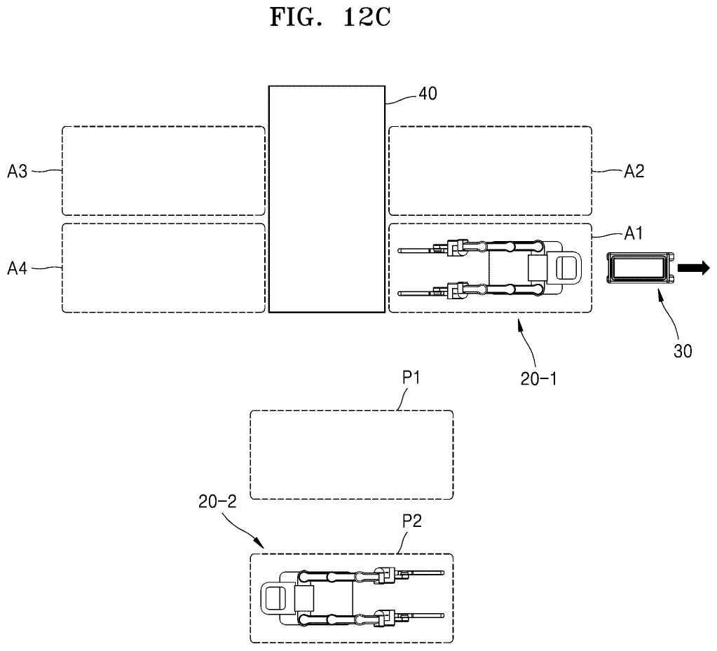

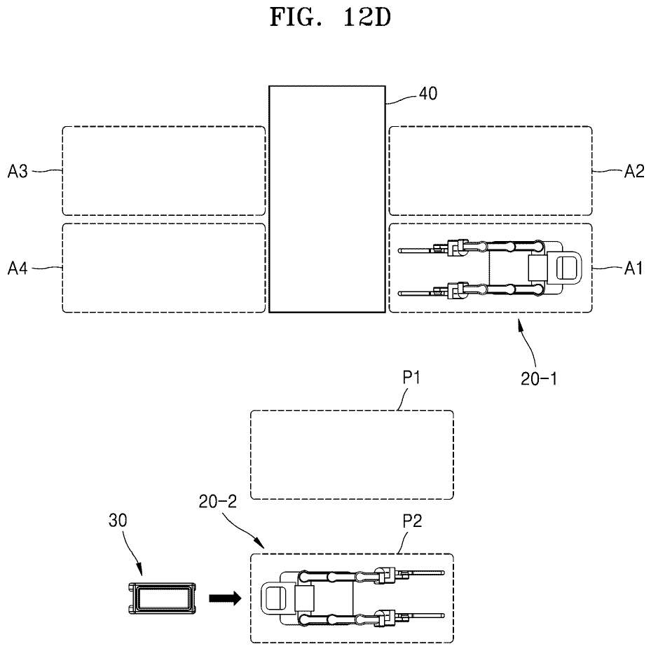

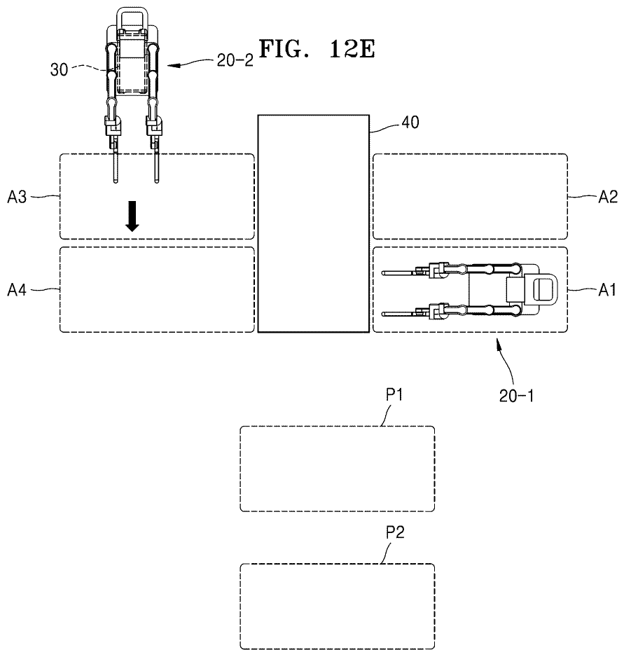

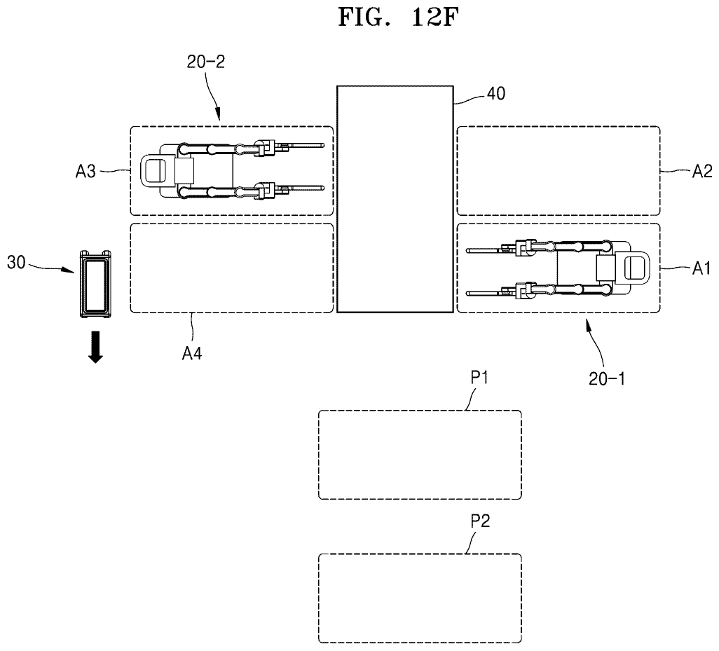

For example, as illustrated in FIGS. 12A to 12F, when the surgical robot system 1 includes two surgical robots 20 and one transport robot 30, a process of selecting the transport robot 30 may not be required, whereas a process of selecting the surgical robot 20 to be transported may be required. In addition, when a plurality of surgical robots 20 are to be transported, a process of selecting the surgical robot 20 to be transported first may also be required.

Thereafter, in operation S211, a placement method for the surgical robot 20 may be selected. Here, the surgical robot 20 may be placed by an automatic placement method or a manual placement method.

In other words, the transporting operation of the surgical robot 20 may include obtaining input information indicating one of automatic placement and manual placement for the surgical robot 20.

For example, a user may select the automatic placement method by manipulating the display member 10b of the master robot 10 or by manipulating the display part 350 of the surgical robot 20. In other words, the control part 15 of the master robot 10 may obtain input information for the automatic placement.

Thereafter, in operation S213, the user may select placement of the surgical robot 20. Specifically, the user may directly select the placement of the surgical robot 20 through a placement diagram displayed on the display member 10b, or may select one of pre-stored placements of the surgical robot 20.

For example, the user may select a type of surgery to be performed by selecting a surgery-type item displayed on the display member 10b, and may select a pre-stored placement of the surgical robot 20 according to the type of surgery.

Here, sub-items for each type of surgery may be composed of various types of surgery cases, and the user may select an appropriate surgery case. In addition, the surgical robot system 1 may store placement information of the surgical robot 20 suitable for a selected type of surgery or a selectable surgical case.

In other words, the placement information of the surgical robot 20 may be stored in the storage part 17 of the master robot 10, and the surgical robot 20 may be placed based on the stored placement information according to input surgery placement selection information.

Thereafter, in operation S214, the transport robot 30 may transport the surgical robot 20 according to the placement selected through the above-described process. Here, the transport robot 30 may transport the surgical robot 20 by performing the above-described autonomous traveling function.

This will be described in detail with reference to FIGS. 12A to 12F. FIGS. 12A to 12F are views schematically illustrating a process in which the surgical robot 20 is placed by the transport robot 30 in the surgical robot system 1 according to an embodiment of the present disclosure.

Referring to FIG. 12A, areas in which surgical robots 20 are to be placed may be provided on opposite sides of the surgical bed 40. For example, a first area A1 and a second area A2 may be provided on one side of the surgical bed 40, and a third area A3 and a fourth area A4 may be provided on another side of the surgical bed 40, but areas in which the surgical robots 20 are placed are not necessarily limited thereto.

In addition, before being placed in the first area A1 to the fourth area A4, the surgical robot 20 may be placed on standby in one area of a surgical space. For example, the first surgical robot 20-1 may be placed in a first parking area P1, and the second surgical robot 20-2 may be placed in a second parking area P2.

Referring to FIG. 12A again, the transport robot 30 first moves toward the first surgical robot 20-1 placed in the first parking area P1. The transport robot 30 may enter below the second main body 320 of the first surgical robot 20-1 and may lift the first surgical robot 20-1 through the lifting module 35a.

Referring to FIG. 12B, the transport robot 30 may move toward the first area A1 in which the first surgical robot 20-1 is to be placed while lifting the first surgical robot 20-1. That is, the transport robot 30 may transport the first surgical robot 20-1.

Referring to FIG. 12C, after transporting the first surgical robot 20-1 to the first area A1, the transport robot 30 may place the first surgical robot 20-1 on a floor surface. That is, the transport robot 30 may place the first surgical robot 20-1 on the floor surface by lowering the lifting module 35a. Thereafter, the transport robot 30 may be disconnected from the first surgical robot 20-1 and may move to another position.

Referring to FIG. 12D, the transport robot 30 moves toward the second surgical robot 20-2 placed in the second parking area P2. The transport robot 30 may enter below the second main body 320 of the second surgical robot 20-2 and may lift the second surgical robot 20-2 through the lifting module 35a.

Referring to FIG. 12E, the transport robot 30 may move toward the third area A3 in which the second surgical robot 20-2 is to be placed while lifting the second surgical robot 20-2. That is, the transport robot 30 may transport the second surgical robot 20-2.

Referring to FIG. 12F, after transporting the second surgical robot 20-2 to the third area A3, the transport robot 30 may place the second surgical robot 20-2 on the floor surface. That is, the transport robot 30 may place the second surgical robot 20-2 on the floor surface by lowering the lifting module 35a. Thereafter, the transport robot 30 may be disconnected from the second surgical robot 20-2 and may move to another position.

In the above description, a case in which only one transport robot 30 is provided and the transport robot 30 sequentially places a plurality of surgical robots 20 has been exemplarily described. When additional transport robots 30 are provided, the transport robots 30 may simultaneously transport different surgical robots 20.

Referring to FIG. 11 again, after the surgical robot 20 is transported, it may be determined whether a change in position of the surgical robot 20 is required (operation S215). In this case, when the surgical robot 20 is not placed at a proper position, the position of the surgical robot 20 may be corrected by manual manipulation (operation S216). That is, the surgical robot 20 may be moved by manual manipulation.

In some embodiments, when placement of the surgical robot 20 is appropriate, the position of the surgical robot 20 may be fixed (operation S220). Specifically, the user may approve the corresponding position by manipulating the display part 350 of the surgical robot 20, or may approve the corresponding position by manipulating the display member 10b of the master robot 10. When the approval is completed, the user may manipulate the fixing part 340 of the surgical robot 20 to fix the surgical robot 20 so as to prevent the surgical robot 20 from moving. Alternatively, the fixing part 340 of the surgical robot 20 may automatically operate in response to a position approval signal to thereby fix the position of the surgical robot 20.

In some embodiments, when the manual placement method is selected in operation S211, the position of the surgical robot 20 may be moved by manual manipulation (operation S212). In this case, the travel assist mode described above may be selected for the transport robot 30. That is, while the transport robot 30 is connected to the surgical robot 20, the transport robot 30 may assist movement of the surgical robot 20 via the travel assist mode. Even when the user directly moves the surgical robot 20 while holding a handle of the surgical robot 20, the user may easily move the surgical robot 20 by operation of the traveling part 32 of the transport robot 30. In addition, even after the surgical robot 20 is placed by manual manipulation, fixing the position of the surgical robot 20 may be performed (operation S220).

In operations S230 to S238, the setup arm 200 of the surgical robot 20 may be set. First, whether a posture change of the setup arm 200 is automatically performed may be selected (operation S231).

In other words, the setting of the setup arm 200 of the surgical robot 20 may include obtaining input information for selecting one of an automatic setting method and a manual setting method for the posture of the setup arm 200.

For example, the user may select the automatic setting method for the setup arm 200 by manipulating the display member 10b of the master robot 10 or the display part 350 of the surgical robot 20. In other words, the control part of the master robot 10 may obtain input information for the automatic setting.

When a setting method of the setup arm 200 is selected as the automatic setting method, in operation S233, the user may select the posture of the setup arm 200. Specifically, the user may directly select the posture of the setup arm 200 through manipulation of the display member 10b, or may select one of postures of the setup arm 200 that are stored in advance.

In other words, when the surgical robot system 1 obtains input information for automatic setting, the surgical robot system 1 may include obtaining selection information for the posture of the setup arm 200 through the display operation.

Thereafter, in operation S234, the setup arm 200 may be changed to a preset posture.

After the setup arm 200 of the surgical robot 20 is set, it may be determined whether a change in the posture of the setup arm 200 is required (operation S235). In addition, after the posture of the setup arm 200 is automatically changed, when a further posture change of the setup arm 200 is required, the posture of the setup arm 200 may be changed through manual manipulation (operation S236).

When a posture change of the setup arm 200 is not required, fine adjustment may be performed through manual manipulation (operation S237).

In some embodiments, when the manual setting method is selected in operation S231, the posture of the setup arm 200 may be changed by manual manipulation (operation S232). In addition, when input information for the manual setting is obtained, the setting of the setup arm 200 of the surgical robot system 1 may further include obtaining brake release information of the setup arm 200. In addition, even after the posture of the setup arm 200 is changed by manual manipulation, fine adjustment may be performed (operation S237).

In an embodiment, the method of transporting and placing the surgical robot 20 may include, after the posture of the setup arm 200 of the surgical robot 20 is changed, resetting the posture of the setup arm 200 or repositioning the surgical robot 20 (operation S238).

That is, when the posture of the setup arm 200 is reset, the method may restart from operation S230, and when the surgical robot 20 is repositioned, the method may restart from operation S210. When resetting the posture of the setup arm 200 or repositioning the surgical robot 20 is not required, the surgical instrument 50 may be mounted on the surgical robot 20 (operation S240).

In other words, after the posture of the setup arm 200 is completely set, the setup arm 200 may be fixed, and the surgical instrument 50 may be mounted on the active arm 100.

According to the present disclosure, it is possible to provide a surgical robot system that can improve ease of moving a surgical robot while allowing the surgical robot to be lightweight, and can automatically place the surgical robot. However, such effects are merely exemplary, and effects of the present disclosure are not limited thereto.

The present disclosure has been described above with a focus on exemplary embodiments. It will be understood by those skilled in the art that various changes in form and details may be made therein without departing from the essential features of the present disclosure. Therefore, the disclosed embodiments should be considered in an illustrative rather than a limiting sense. The scope of the present disclosure is defined not by the detailed description of the disclosure but by the appended claims, and all differences within the scope will be construed as being included in the present disclosure.

What is claimed is:

1. A surgical robot system comprising:
a surgical robot including at least one arm unit;
a transport robot operable to transport the surgical robot; and
a master robot including a manipulation member configured to control a motion of the transport robot,
wherein the transport robot moves toward the surgical robot in response to a received signal and places the surgical robot at a position corresponding to the received signal.

2. The surgical robot system of claim 1, wherein the transport robot includes:
a traveling part configured to move or stop a body of the transport robot;
a surgical robot connection part configured to be brought into contact with and connected to the surgical robot; and
a detection part configured to detect a surrounding environment of the transport robot and assist a travel of the transport robot.

3. The surgical robot system of claim 2, wherein
the surgical robot connection part includes a lifting module configured to raise and lower the surgical robot, and
the transport robot is further operable to transport the surgical robot while the surgical robot is lifted by the lifting module.

4. The surgical robot system of claim 2, wherein
the surgical robot connection part includes a docking module configured to be connected to the surgical robot, and
the transport robot is further operable to move the surgical robot by pulling or pushing the surgical robot while the transport robot is connected to the surgical robot via the docking module.

5. The surgical robot system of claim 2, wherein the detection part is operable to detect the surgical robot in a vicinity of the transport robot.

6. The surgical robot system of claim 2, wherein the detection part is operable to detect a surgical bed positioned in a surgical space.

7. The surgical robot system of claim 2, wherein the transport robot further includes a power supply part configured to supply a power to the surgical robot.

8. The surgical robot system of claim 1, further comprising a surgical bed including a sensor configured to transmit position information of the surgical bed to at least one of the surgical robot, the transport robot, and the master robot.

9. The surgical robot system of claim 1, wherein the master robot is operable to control a motion of the surgical robot.

10. The surgical robot system of claim 1, wherein the transport robot is further operable to autonomously travel in an operating room.

11. The surgical robot system of claim 1, wherein the transport robot places the surgical robot at a preset position in response to the received signal.

12. The surgical robot system of claim 11, wherein
position information indicating a position at which the surgical robot is placed is stored while being categorized according to a type of surgery, and
the transport robot places the surgical robot according to a type of surgery selected among stored types of surgery.

13. The surgical robot system of claim 1, further comprising an autonomous traveling mode and a travel assist mode among selectable driving modes for the transport robot.

14. The surgical robot system of claim 13, wherein when the autonomous traveling mode is selected, the transport robot performs an autonomous traveling function to move toward the surgical robot, is connected to the surgical robot, and places the surgical robot at a preset position.

15. The surgical robot system of claim 13, wherein when the travel assist mode is selected, the transport robot detects a force transmitted thereto from outside to drive the traveling part.

16. A transport robot for transporting a surgical robot, comprising:
a traveling part configured to move or stop a body of the transport robot;
a surgical robot connection part configured to be brought into contact with and connected to the surgical robot; and
a detection part configured to detect a surrounding environment of the transport robot and assist a travel of the transport robot,
wherein the transport robot moves toward the surgical robot in response to a received signal and places the surgical robot at a position corresponding to the received signal.

17. The transport robot of claim 16, wherein the transport robot is operable to receive position information of a surgical bed from the surgical bed.

18. A method of transporting and placing a surgical robot using a transport robot, the method comprising:
transporting the surgical robot by the transport robot;
fixing a position of the surgical robot;
setting a setup arm of the surgical robot; and
mounting a surgical instrument on the surgical robot.

19. The method of claim 18, wherein the transporting of the surgical robot includes selecting the surgical robot to be transported and the transport robot configured to transport the selected surgical robot.

20. The method of claim 18, wherein the transporting of the surgical robot includes obtaining input information indicating one of automatic placement and manual placement for the surgical robot.

21. The method of claim 20, further comprising obtaining selection information for placement of the surgical robot through a display operation, when the input information indicating the automatic placement is obtained.

22. The method of claim 20, further comprising allowing the surgical robot to be moved under user control when the input information indicating the manual placement is obtained.

23. The method of claim 18, wherein the transporting of the surgical robot includes transporting the surgical robot to a preset position by the transport robot that autonomously travels.

24. The method of claim 18, wherein the transporting of the surgical robot includes allowing the surgical robot to be moved through manual manipulation when a change in the position of the surgical robot is required after the surgical robot is transported by the transport robot.

25. The method of claim 18, wherein the fixing of the position of the surgical robot includes operating a fixing part of the surgical robot in response to an approval signal received via a robot display to fix the position of the surgical robot.

26. The method of claim 18, wherein the setting of the setup arm of the surgical robot includes obtaining input information indicating one of automatic setting and manual setting for a posture of the setup arm.

27. The method of claim 26, further comprising obtaining selection information for the posture of the setup arm through a display operation when the input information indicating the automatic setting is obtained.

28. The method of claim 26, further comprising obtaining brake release information of the setup arm when the input information indicating the manual setting is obtained.

29. The method of claim 18, wherein the setting of the setup arm of the surgical robot includes changing the setup arm to a preset posture.

30. The method of claim 18, wherein in the setting of the setup arm of the surgical robot, when a change in a posture of the setup arm is required after the posture of the setup arm is automatically changed, the posture of the setup arm is adjustable through manual manipulation.

31. The method of claim 18, further comprising resetting a posture of the setup arm or resetting the position of the surgical robot, after the posture of the setup arm of the surgical robot is changed.
